(19) United States
(12) Patent Application Publication
(10) Pub. No.: US 2025/0197893 A1
(43) Pub. Date: Jun. 19, 2025

(54) ANAEROBIC DIGESTION OF AT LEAST ONE STILLAGE COMPOSITION, AND RELATED SYSTEMS

(71) Applicant: POET Research, Inc., Sioux Falls, SD (US)

(72) Inventors: Brady Christopher Clavel, Yankton, SD (US); Benjamin M. Gerdes, Yankton, SD (US); Anna M. Andersen, Valley Springs, SD (US); Amber Jerke, Yankton, SD (US)

(21) Appl. No.: 18/977,801

(22) Filed: Dec. 11, 2024

Related U.S. Application Data
(60) Provisional application No. 63/610,964, filed on Dec. 15, 2023.

(51) Int. Cl. C12P 5/02; C12M 1/00; C12M 1/107

(52) U.S. Cl. CPC C12P 5/023; C12M 21/04; C12M 45/04

(57) ABSTRACT

Methods and systems for using one or more stillage compositions generated at a bioprocessing facility for anaerobic digestion. Two or more anaerobic digestion digesters are configured with respect to each other based at least on the suspended solids content and soluble solids content of each process stream that is fed to each anaerobic digestion digester.

### CROSS-REFERENCE TO RELATED APPLICATION

This nonprovisional patent application claims the benefit of U.S. Provisional Patent Application Ser. No. 63/610,964, filed on Dec. 15, 2023, wherein said provisional patent application is incorporated herein by reference in its entirety.

### BACKGROUND

The present disclosure relates to methods and systems for using one or more stillage compositions generated at a bioprocessing facility for anaerobic digestion. There is a continuing need for improved methods and systems for using one or more stillage compositions generated at a bioprocessing facility for anaerobic digestion.

### SUMMARY

Embodiments of the present disclosure include a method of producing biogas from one or more stillage streams. The method includes separating a stillage composition into at least a first fraction and a second fraction; exposing at least a portion of the first fraction to anaerobic digestion conditions in at least one first anaerobic digestion digester to form an anaerobic digestion digestate composition; separating at least a portion of the anaerobic digestion effluent from the at least one first anaerobic digestion digester into at least anaerobic digestion liquid effluent and anaerobic digestion solid effluent; exposing at least a portion of the second fraction to anaerobic digestion conditions in at least one second anaerobic digestion digester to form an anaerobic digestion digestate composition; introducing at least a portion of the anaerobic digestion liquid effluent from the at least one first anaerobic digestion digester to the at least one second anaerobic digestion digester; and exposing the at least a portion of the anaerobic digestion liquid effluent from the at least one first anaerobic digestion digester to anaerobic digestion conditions in the at least one second anaerobic digestion digester to form the anaerobic digestion digestate composition. The first fraction has a suspended solids content greater than a suspended solids content of the second fraction. The anaerobic digestion digestate composition comprises biogas and anaerobic digestion effluent. The anaerobic digestion solid effluent has a suspended solids content greater than a suspended solids content of the anaerobic digestion liquid effluent. The anaerobic digestion digestate composition includes biogas and anaerobic digestion effluent. The at least one second anaerobic digestion digester and the at least one first anaerobic digestion digester are not the same anaerobic digestion digester.

Embodiments of the present disclosure also include a bioprocessing facility that includes a source of a stillage composition; a separation system in fluid communication with the source of the stillage composition; at least one first anaerobic digestion digester in fluid communication with at least a portion of the first fraction; a separation system in fluid communication with at least a portion of the anaerobic digestion effluent from the at least one first anaerobic digestion digester; and at least one second anaerobic digestion digester in fluid communication with at least a portion of the second fraction. The separation system is configured to separate the stillage composition into at least a first fraction and a second fraction. The first fraction has a suspended solids content greater than a suspended solids content of the second fraction. The at least one first anaerobic digestion digester is configured to receive the at least a portion of the first fraction and expose the at least a portion of the first fraction to anaerobic digestion conditions to form an anaerobic digestion digestate composition. The anaerobic digestion digestate composition includes biogas and anaerobic digestion effluent. The separation system is configured to receive the at least a portion of the anaerobic digestion effluent and separate the at least a portion of the anaerobic digestion effluent from the at least one first anaerobic digestion digester into at least anaerobic digestion liquid effluent and anaerobic digestion solid effluent. The anaerobic digestion solid effluent has a suspended solids content greater than a suspended solids content of the anaerobic digestion liquid effluent. The at least one second anaerobic digestion digester is configured to receive the at least a portion of the second fraction and expose the at least a portion of the second fraction to anaerobic digestion conditions to form an anaerobic digestion digestate composition. The anaerobic digestion digestate composition includes biogas and anaerobic digestion effluent. The at least one second anaerobic digestion digester and the at least one first anaerobic digestion digester are not the same anaerobic digestion digester. At least a portion of the anaerobic digestion liquid effluent from the at least one first anaerobic digestion digester is in fluid communication with the at least one second anaerobic digestion digester. The at least one second anaerobic digestion digester is configured to receive and expose the at least a portion of the anaerobic digestion liquid effluent from the at least one first anaerobic digestion digester to anaerobic digestion conditions to form the anaerobic digestion digestate composition.

Embodiments of the present disclosure also include a method of producing biogas from one or more stillage streams, where the method includes exposing at least one stillage composition to anaerobic digestion conditions in at least one first anaerobic digestion digester (low organic loading digester) to form an anaerobic digestion digestate composition; separating at least a portion of the anaerobic digestion effluent from the at least one first anaerobic digestion digester into at least anaerobic digestion liquid effluent and anaerobic digestion solid effluent; exposing at least a portion of the anaerobic digestion liquid effluent to anaerobic digestion conditions in at least one second anaerobic digestion digester to form an anaerobic digestion digestate composition; exposing at least a portion of the anaerobic digestion solid effluent to anaerobic digestion conditions in at least one third anaerobic digestion digester to form an anaerobic digestion digestate composition; separating at least a portion of the anaerobic digestion effluent from the at least one third anaerobic digestion digester into at least anaerobic digestion liquid effluent and anaerobic digestion solid effluent; introducing at least a portion of the anaerobic digestion liquid effluent from the at least one third anaerobic digestion digester to the at least one second anaerobic digestion digester; and exposing the at least a portion of the anaerobic digestion liquid effluent from the at least one third anaerobic digestion digester to anaerobic digestion conditions in the at least one second anaerobic digestion digester to form the anaerobic digestion digestate composition. The anaerobic digestion digestate composition includes biogas and anaerobic digestion effluent. The anaerobic digestion solid effluent has a suspended solids content greater than a suspended solids content of the anaerobic digestion liquid effluent. The anaerobic digestion digestate composition includes biogas and anaerobic digestion effluent. The first anaerobic digestion digester, the second anaerobic digestion digester, and the third anaerobic digestion digester are not the same anaerobic digestion digester. The anaerobic digestion solid effluent has a suspended solids content greater than a suspended solids content of the anaerobic digestion liquid effluent.

Embodiments of the present disclosure also include a bioprocessing facility that includes a source of a stillage composition; at least one first anaerobic digestion digester in fluid communication with at least a portion of the stillage composition; a separation system in fluid communication with at least a portion of the anaerobic digestion effluent from the at least one first anaerobic digestion digester; at least one second anaerobic digestion digester in fluid communication with at least a portion of the anaerobic digestion liquid effluent from the separation system; at least one third anaerobic digestion digester in fluid communication with at least a portion of the anaerobic digestion solid effluent from the separation system; and a separation system in fluid communication with at least a portion of the anaerobic digestion effluent from the at least one third anaerobic digestion digester. The at least one first anaerobic digestion digester is configured to receive the at least a portion of the stillage composition and expose the at least a portion of the stillage composition to anaerobic digestion conditions to form an anaerobic digestion digestate composition. The anaerobic digestion digestate composition comprises biogas and anaerobic digestion effluent. The separation system is configured to receive the at least a portion of the anaerobic digestion effluent and separate the at least a portion of the anaerobic digestion effluent from the at least one first anaerobic digestion digester into at least anaerobic digestion liquid effluent and anaerobic digestion solid effluent. The anaerobic digestion solid effluent has a suspended solids content greater than a suspended solids content of the anaerobic digestion liquid effluent. The at least one second anaerobic digestion digester is configured to receive the at least a portion of the anaerobic digestion liquid effluent and expose the at least a portion of the anaerobic digestion liquid effluent to anaerobic digestion conditions to form an anaerobic digestion digestate composition that includes biogas and anaerobic digestion effluent. The at least one third anaerobic digestion digester is configured to receive the at least a portion of the anaerobic digestion solid effluent and expose the at least a portion of the anaerobic digestion solid effluent to anaerobic digestion conditions to form an anaerobic digestion digestate composition that includes biogas and anaerobic digestion effluent. The at least one first anaerobic digestion digester, the at least one first anaerobic digestion digester, and the at least one first anaerobic digestion digester are not the same anaerobic digestion digester. The separation system is configured to receive the at least a portion of the anaerobic digestion effluent and separate the at least a portion of the anaerobic digestion effluent from the at least one third anaerobic digestion digester into at least anaerobic digestion liquid effluent and anaerobic digestion solid effluent that has a suspended solids content greater than a suspended solids content of the anaerobic digestion liquid effluent. At least a portion of the anaerobic digestion liquid effluent from the at least one third anaerobic digestion digester is in fluid communication with the at least one second anaerobic digestion digester. The at least one second anaerobic digestion digester is configured to receive and expose the at least a portion of the anaerobic digestion liquid effluent from the at least one third anaerobic digestion digester to anaerobic digestion conditions to form the anaerobic digestion digestate composition.

### BRIEF DESCRIPTION OF THE DRAWINGS

Various examples of the present disclosure will be discussed with reference to the appended drawings. These drawings depict only illustrative examples of the disclosure and are not to be considered limiting of its scope.

FIG. 1 shows a process-flow diagram of a non-limiting embodiment of a bioprocessing facility configured to separate ethanol from beer and form whole stillage and additional stillage compositions as described herein below;

FIG. 2 shows a process-flow diagram of a non-limiting embodiment of a bioprocessing facility configured to use stillage compositions for anaerobic digestion; and

FIG. 3 shows a process-flow diagram of another non-limiting embodiment of a bioprocessing facility configured to use stillage compositions for anaerobic digestion.

### DETAILED DESCRIPTION

The present disclosure relates to methods and systems for using one or more stillage compositions generated at a bioprocessing facility for anaerobic digestion. According to the present disclosure, two or more anaerobic digestion digesters are configured with respect to each other based at least on the suspended solids content of each process stream that is fed to each anaerobic digestion digester. Managing the feed streams in this manner can advantageously make the overall system more efficient; more compact in terms of overall space that is used for the anaerobic digestion digesters; and/or manage water balance of the bioprocessing facility.

As used herein, a bioprocessing facility refers to a facility that can produce one or more bioproducts by converting biomass feedstock via one or more physical processes, one or more chemical processes, one or more bioprocesses, and combinations thereof. Non-limiting examples of bioprocessing facilities include dry mills, wet mills, biofuel production facilities, soy processing facilities, and the like. A bioproduct refers to a product derived from a biological, renewable resource. For example, a bioproduct can be a component of biomass feedstock that is liberated from the biomass feedstock (e.g., corn oil from corn grain) and/or can include a chemical (“biochemical” or “target biochemical”) that is produced by a biocatalyst (e.g., microorganism and/or enzyme) such as, for example, alcohol produced by yeast fermenting sugar. Non-limiting examples of bioproducts produced in a bioprocessing facility include one or more of fuel, feed, food, pharmaceuticals, beverages and precursor chemicals. In some embodiments, a bioproduct includes, among others, one or more monomeric sugars, one or more enzymes, one or more oils, one or more alcohols (e.g., ethanol, butanol, and the like), fungal biomass, amino acids, and one or more organic acids (e.g., lactic acid), and combinations thereof.

As used herein, a “stillage composition” refers to a back-end composition of a fermentation process after separating one or more bioproducts from beer to form at least one target bioproduct stream and one or more co-product streams. The one or more bioproducts can be separated from beer using one or more separation technologies such as membrane separation, gas stripping, absorption, distillation, and the like. A stillage composition can include whole stillage, at least one stillage composition derived from whole stillage, and combinations thereof. Non-limiting examples of a stillage composition derived from whole stillage include thin stillage, concentrated thin stillage (syrup), defatted syrup, defatted emulsion, clarified thin stillage, distiller's oil, distiller's grain, distiller's yeast, and the like. Defatted syrup and defatted emulsion are examples of stillage compositions that remain after fat (e.g., corn oil) has been separated from syrup and emulsion, respectively, and each can be referred to as a “defatted stillage composition.” Non-limiting examples of methods and systems for processing stillage streams are also described in U.S. Pat. No. 8,702,819 (Bootsma); U.S. Pat. No. 9,061,987 (Bootsma); U.S. Pat. No. 9,290,728 (Bootsma); U.S. Pat. No. 10,059,966 (Bootsma); U.S. Pat. No. 11,248,197 (Bootsma); and U.S. Pub. No. 2020/0140899 (Bootsma); wherein the entirety of each of said patent documents is incorporated herein by reference.

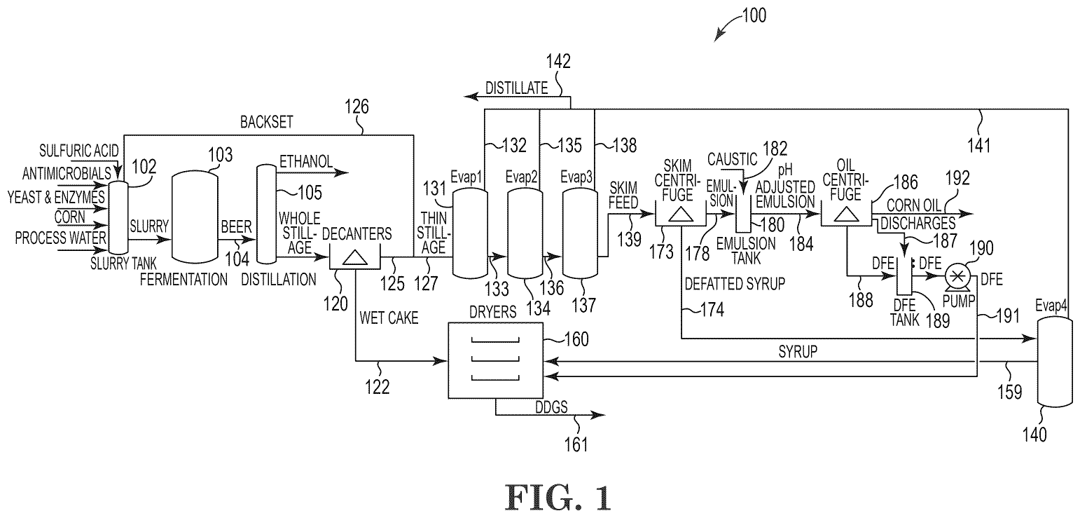

For illustration purposes, a non-limiting example of a bioprocessing facility that produces one or more stillage compositions will be described with respect to FIG. 1. FIG. 1 illustrates an example of dry-grind corn ethanol biorefinery 100 (“biorefinery 100”) as a bioprocessing facility that produces stillage compositions described above. The biorefinery 100 includes a “front end” and a “back end.” The front end includes distillation system 105 and upstream from distillation system 105. As shown in FIG. 1, the front end starts with adding one or more materials to a slurry tank 102 to form a fermentable composition that can ferment in front-end fermentation system 103. Non-limiting examples of materials used to form a fermentable composition include one or more of microorganisms, enzymes, carbon sources (e.g., feedstock), aqueous compositions (e.g., fresh water, backset, etc.), nutrient sources (e.g., feedstock), etc.

In some embodiments, a feedstock can function as a carbon source and/or a nutrient source, and can be used to form a fermentable composition. A feedstock can include one or more components that are utilized by a microorganism to produce one or more bioproducts via a bioprocess. Non-limiting examples of a feedstocks can be derived from biomass (e.g., plant-based) and may include, e.g., monosaccharides such as glucose and fructose, disaccharides such as sucrose and lactose, and more complex polysaccharides such as starch, cellulose, hemicellulose, and pectin. These biomass-derived feedstocks may come from the seed, sap, stems, and leaves of plants. A wide variety of plant-based feedstocks can be used according to the present disclosure such as sugar beets, sugar cane, grains, legumes, crop residues (e.g., husks, stems, corn stover, sugarcane bagasse, wheat straw), grasses, and woody plants. In some embodiments, feedstock can be derived from corn, sorghum, wheat, rice, barley, soybean, rapeseed, oats, millet, rye, corn stover, straw, bagasse and the like. In some embodiments, as shown in FIG. 1, a feedstock can include whole ground grain (e.g., corn flour) formed via, e.g., a dry-grind process.

In some embodiments, a bioprocessing facility can include one or more feedstock systems to process feedstock from one form into another form prior to fermentation. For example, a feedstock system can include one or more size-reduction devices to reduce the size of raw feedstock such as grain and/or further reducing the size of ground grain that has previously been reduced in size. Methods for reducing the size of feedstock, e.g., corn and/or previously ground corn, into fine particles prior to fermentation include dry methods such as passing corn through one or more hammer mills, ball mills and/or roller mills or wet methods such as passing a ground grain slurry through one or more mills such as a disc mill, roller mill, colloid mill, ball mill or other type of milling device.

In some embodiments, a ground feedstock can be mixed with an appropriate amount of water (e.g., in a slurry tank 102) to form at least a portion of a fermentable composition (sometimes referred to as a mash). In some embodiments, whole ground corn can be mixed with liquid at about 20 to about 50 wt-% or about 25 to about 45 wt-% dry whole ground corn based on the total weight of the slurry. Whole ground corn can include starch, fiber, protein, oil, endogenous enzymes, amino acids, etc. As shown in FIG. 1, whole ground corn is mixed in slurry tank 102 with process water.

One or more exogenous microorganisms can be present in the fermentable composition of the fermentation system 103 to produce beer that includes at least one or more biochemical bioproducts. A bioproduct refers to a product derived from a biological, renewable resource. For example, a bioproduct can be a component of biomass feedstock that is liberated from the biomass feedstock (e.g., corn oil from corn grain) and/or can include a chemical (“biochemical”) that is produced by a biocatalyst (e.g., microorganism and/or enzyme) such as, for example, alcohol produced by yeast fermenting sugar. Fermentation by a microorganism can produce biomass (e.g., single cell protein (SCP)), extracellular metabolites (e.g., alcohol such as ethanol), intracellular metabolites (e.g., enzymes), and combinations thereof. Non-limiting examples of such microorganisms include one or more of ethanologens, butanologens, and the like. Exemplary microorganisms include one or more of yeast, algae, or bacteria. For example, yeast may be used to convert the sugars to an alcohol such as ethanol. Suitable yeast includes any variety of commercially available yeast, such as commercial strains of Saccharomyces cerevisiae. As shown in FIG. 1, yeast is added as a microorganism to slurry tank 102 to form a fermentable composition.

Optionally, one or more additional, exogenous materials may be utilized in a fermentable composition. Non-limiting examples of such materials include one or more of enzymes, pH adjusters, antimicrobials (e.g., used against bacterial contaminants in yeast fermentation), and the like. As shown in FIG. 1, sulfuric acid is added to slurry tank 102 to adjust the pH, one or more antimicrobials are added to slurry tank to act against bacterial contaminants in yeast fermentation, and one or more exogenous enzymes are added discretely to slurry tank 102 to help break corn starch into monosaccharide such as glucose. Optionally or alternatively, one or more enzymes may be used for hydrolysis can be produced by one or more microorganisms present in a fermentable composition.

A front-end fermentation system according to the present disclosure can include one or more vessels that are adapted to expose a fermentable composition to conditions suitable for converting monosaccharide such as glucose to one or more bioproducts. As used herein, a “vessel” refers to any vessel that permits a bioproduct to be formed from a microorganism via fermentation. In some embodiments, a vessel can refer to a bioreactor adapted or configured to expose a fermentable composition to fermentation conditions. Non-limiting examples of vessels that can expose a fermentable composition to fermentation conditions include fermenters, beer wells, and the like. Two or more vessels may be arranged in any desired configuration such as parallel or series. As shown in FIG. 1, front-end fermentation system 103 includes a single fermenter.

A front-end fermentation system is configured to expose fermentable composition to fermentation conditions so that one or more microorganisms can convert one or more components in the fermentable composition such as sugars into a beer that includes one or more target bioproducts. Fermentation conditions include one or more conditions such as pH, time, temperature, aeration, stirring, and the like.

The pH of a fermentable composition can be at a pH that helps a microorganism produce one or more target bioproducts in a desired quantity. In some embodiments, the pH is greater than 3.5, e.g., from 3.5 to 7, from 3.5 to 5.5, or even from 3.5 to 4.5. Techniques for adjusting and maintaining pH include, e.g., adding one or more acidic materials and/or adding one or more basic materials. As mentioned above, sulfuric acid is added to slurry tank 102 to adjust the pH.

With respect to temperature and time, the contents of a fermentable composition can be maintained at temperature for time period helps a microorganism produce one or more target bioproducts in a desired quantity. In some embodiments, the temperature of a fermentable composition can be at a temperature in a range from 20° C. to 45° F., from 25° C. to 40° C., or even from 30° C. to 40° C. In some embodiments, front-end fermentation can occur for a time period up to 72 hours, up to 80 hours, up to 90 hours, up to 100 hours, or even longer. In some embodiments, front-end fermentation can occur for a time period from 40 hours to 100 hours, from 50 hours to 90 hours, or even from 60 hours to 85 hours.

Front-end fermentation can be performed under anaerobic conditions and/or aerobic conditions. For example, front-end fermentation can be performed under aerobic conditions for at least a portion of the fermentation and performed under anaerobic conditions for another portion of fermentation. Alternatively, all of fermentation can be performed under anaerobic conditions or under aerobic conditions. Anaerobic or aerobic conditions are selected based on the “target” biochemical or biochemicals chosen to be produced by a microorganism even though there may de minimis amounts of “non-target” biochemicals that are also produced by the microorganism. Anaerobic conditions means that the fermentation process is conducted without any intentional introduction of oxygen-containing gases such as with equipment like blowers, compressors, etc., that could operate to create an aerobic environment suitable for aerobic fermentation. It is noted that while simply stirring a fermentable composition to keep reactor contents homogenous may or may not introduce a de minimis amount of an oxygen-containing gas such as air in some embodiments, stirring alone may not create conditions that would be considered “aerobic conditions” as used herein. However, if desired, the contents of a fermenter could be mixed using appropriate equipment such that sufficient oxygen is introduced throughout the fermentable composition to create an aerobic environment suitable for aerobic fermentation (see below).

Aerobic conditions means that fermentation is performed with intentional introduction of one or more oxygen-containing gasses (“aeration”) to create an aerobic environment suitable for aerobic fermentation so that oxygen can be consumed by one or more microorganisms and selectively favor the production of enzymes via an aerobic metabolic pathway as compared to an anaerobic pathway which favors production of biochemicals (e.g., alcohol, organic acids, and the like). A fermentation system may incorporate aeration by including one or more blowers, spargers, gas compressors, mixing devices, and the like, that are in fluid communication with one or more fermentation vessels and that can introduce an oxygen-containing gas (e.g., air) into a fermentable composition during at least a portion of fermentation. For example, an oxygen- containing gas can be sparged into a fermentable composition so that the gas bubbles up and through the fermentable composition and oxygen transfers into the fermentable composition. As another example, an oxygen-containing gas can be introduced into the headspace of a fermenter so that the gas diffuses into the fermentable composition.

In some embodiments, an aerobic fermentation can be quantified by referring to a volumetric oxygen transfer coefficient (“kLa” constant) (hours(h)−1). The kLa constant describes how efficient oxygen is transferred from gas bubbles into the fermentable composition. The kLa constant depends on factors such as process conditions and geometry of a vessel used for fermentation (e.g., a fermenter). Process conditions include the volume flow of oxygen in the form of gas into a fermentable composition, pressure of the contents of a vessel, temperature of the contents of a vessel, and/or degree of mixing of the contents of a vessel. Geometry of a vessel used for fermentation includes height of the vessel. The kLa constant consists of the two coefficients. The mass transfer coefficient “kL,” which describes the transport of oxygen and gas into the liquid phase. And “a,” which refers to the gas-liquid exchange area per unit of liquid volume. Since it can be difficult to measure the kL and a value separately, they are combined into one parameter, the kLa constant. There are chemical, biological and physical methods that measure the kLa constant in a vessel used for fermentation. One method is referred to as the “static gassing-out” method, which involves installing an oxygen sensor in a vessel used for fermentation to measure the dissolved oxygen concentration in a liquid medium. The characterization is often done with water, but any liquid medium can be used. The oxygen concentration of the liquid medium is set to zero by degassing with nitrogen. Then, oxygen containing gas is introduced or “gassed” (e.g., sparged) into the contents of the vessel again under process conditions using a defined gassing rate and stirrer speed. The oxygen sensor then measures the saturation process and the kLa can be determined. In some embodiments, a fermentation vessel operating under aerobic conditions has a kLa constant greater than 0.2, greater than 0.25 or even greater than 0.3 (e.g., from 0.3 to 5, or even from 0.35 to 3).

Optionally, in addition to aeration, a fermentable composition can be agitated or mixed to facilitate transferring oxygen into and throughout the fermentable composition so as to achieve an aerobic environment. For example, a continuous stirred tank reactor (CSTR) can be used to agitate or mix the fermentable composition. The speed of the stirring mechanism (rpms) can be adjusted based on a variety of factors such as tank size, viscosity, and the like. As mentioned above, in addition to mixing the contents of a composition, mixing can be selected, if desired, to intentionally incorporate oxygen to a fermentable composition to facilitate aerobic fermentation.

A front-end fermentation system can be operated according to batch fermentation, fed-batch fermentation, or continuous fermentation (continuous feed and discharge from a vessel such as a fermenter).

Also, a front-end fermentation system according to the present disclosure can conduct fermentation sequentially or simultaneously with respect to a polysaccharide hydrolysis/saccharification process (e.g., jet-cooking and/or enzymatic hydrolysis). Saccharification and fermentation can occur simultaneously according to what is known as “simultaneous saccharification and fermentation” (“SSF”). Sequential hydrolysis and fermentation can also be referred to as separate hydrolysis and fermentation (SHF).

An example of an SSF is described below in the context of a starch-containing grain such as corn. A slurry (“grain mash composition”) can be combined with a microorganism to form a fermentable composition so that at least a portion of starch in the fermentable composition is hydrolyzed by one or more enzymes to produce monosaccharides. As the monosaccharides are produced, they can be metabolized by a microorganism into a target biochemical product. For example, sugar (glucose, xylose, mannose, arabinose, etc.) that is generated from saccharification can be fermented into one or more biochemicals (e.g., butanol, ethanol, and the like).

Alternatively, an SHF process may include a dedicated saccharification process that is separate from a fermentation process (either in the same or separate vessel). For example, after forming an aqueous slurry that includes the biomass feedstock (e.g. corn material from a milling system) the aqueous slurry can be subjected to saccharification in one or more slurry tanks to break down (hydrolyze) at least a portion of the polysaccharides, e.g. starch, cellulose, hemicellulose, etc., into oligosaccharides and/or monosaccharides (e.g. glucose, xylose, mannose, arabinose, etc.) that can be used by microorganisms (e.g., yeast) in a subsequent fermentation process.

Saccharification can be performed by a variety of mechanisms. For example, heat and/or one or more enzymes can be used to form one or more monosaccharides by saccharifying one or more oligosaccharides and/or one or more polysaccharides that are present in a polysaccharide such as starch. In some embodiments, a relatively low temperature saccharification process (whether used in SSF or SHF) involves enzymatically hydrolyzing at least a portion of starch in an aqueous slurry at a temperature below starch gelatinization temperatures, so that saccharification occurs directly from the raw native insoluble starch to soluble glucose while bypassing conventional starch gelatinization conditions, which are typically in a range of 57° C. to 93° C. depending on the starch source and polysaccharide type. In some embodiments, saccharification includes using one or more enzymes (e.g., alpha-amylases and/or gluco-amylases) to enzymatically hydrolyze at least a portion of the starch in the aqueous slurry at a temperature of 40° C. or less to produce a slurry that includes glucose. In some embodiments, enzymatic hydrolysis occurs at a temperature in the range of from 25° C. to 35° C. to produce a slurry that includes glucose. Non-limiting examples of converting starch to glucose are described in U.S. Pat. Nos. 7,842,484 (Lewis), 7,919,289 (Lewis), 7,919,291 (Lewis et al.), 8,409,639 (Lewis et al.), 8,409,640 (Lewis et al.), 8,497,082 (Lewis), 8,597,919 (Lewis), 8,748,141 (Lewis et al.), 2014-0283226 (Lewis et al.), and 2018-0235167 (Lewis et al.), wherein the entirety of each patent document is incorporated herein by reference.

After fermentation, one or more bioproducts can be separated from beer to form at least one target bioproduct stream (e.g., ethanol) and one or more co-product streams (e.g., whole stillage). A separation system according to the present disclosure can be configured to separate at least a portion of at least one of the one or more bioproducts produced in the fermentation system 103. Referring to FIG. 1, an exemplary separation system is illustrated as a distillation system 105 in fluid communication with the fermentation system 103 to receive a beer, which is separated into ethanol as a target biochemical and whole stillage. Whole stillage includes protein (e.g., plant protein (e.g., corn protein) and/or yeast protein from spent yeast cells), oil, fiber, residual carbohydrate (e.g., cellulose, starch, and the like), vitamins, minerals, and combinations thereof.

Whole stillage can be separated into thin stillage and wet cake using one or more solid-liquid separators. Non-limiting examples of solid-liquid separators include one or more centrifuges (e.g., two-phase vertical disk-stack centrifuge, three-phase vertical disk-stack centrifuge, filtration centrifuge), one or more decanters (e.g., filtration decanters), one or more filters (e.g., fiber filter, rotary vacuum drum filter, filter device having one or more membrane filters), one or more screen devices ((e.g., a “DSM” screen, which refers to a Dutch State Mines screen or sieve bend screen (also “gravity” screen), and is a curved concave wedge bar type of stationary screen); one or more pressure screens; one or more paddle screens; one or more rotary drum screens; one or more centrifugal screeners; one or more linear motion screens; one or more vacu-deck screen; etc.), one or more brush strainers, one or more vibratory separators, one or more hydrocyclones, and combinations thereof. As shown in FIG. 1, whole stillage is fed to multiple decanters 120 in parallel to separate whole stillage into wet cake 122 and thin stillage 125. The wet cake 122 is dried in dryer system 160 to form dried distillers' grain (DDGS) 161.

A portion 126 of the thin stillage 125 is transferred to the slurry tank 102 as backset, while the rest 127 of the thin stillage 125 is transferred to an evaporation train that may include 4 to 8 evaporators in series (depending on plant size) to remove water and form syrup. As shown in FIG. 1, the evaporation train includes four evaporators 131, 134, 137, and 140. The rest 127 of the thin stillage 125 is first fed to evaporator 131 to separate moisture as water vapor stream 132 and form a concentrated thin stillage 133. The concentrated thin stillage 133 is fed to evaporator 134 to separate moisture as water vapor stream 135 and form a concentrated thin stillage 136. The concentrated thin stillage 136 is fed to evaporator 137 to separate moisture as water vapor stream 138 and form a semi-concentrated syrup 139. Prior to reaching the end of the evaporator train the semi-concentrated syrup 139 (“skim feed”) is sent to a corn oil separation system, which removes corn oil product 192.

First, the semi-concentrated syrup 139 is separated in a “skim” centrifuge 173 into an emulsion 178 and defatted syrup 174. The defatted syrup 174 is sent to the final evaporator 140 in the evaporator train to separate moisture as water vapor stream 141 to form syrup 159, which is sent to dryer system 160 along with wet cake 122 to form DDGS 161. The emulsion 178 is combined with caustic 182 in emulsion tank 180 to help “break” the emulsion into an oil phase and aqueous phase that are more easily separated from each other. The treated emulsion 184 is pumped to oil centrifuge 186, wherein the treated emulsion 184 is separated into a corn oil product 192 and defatted emulsion (DFE) 188. The defatted emulsion 188 can accumulate in defatted emulsion (DFE) tank 189 and defatted emulsion 191 can be pumped via pump 190 to any desired location. As shown in FIG. 1, the defatted emulsion 191 is sent to dryer system 160 along with wet cake 122 and syrup 159 to form DDGS 161. Also, all of the water vapor streams 132, 135, 138 and 141 are combined into a single distillate stream 142.

The skim centrifuge 173 and oil centrifuge 186 can be centrifuges such as disk-stack centrifuges. In some embodiments, the skim centrifuge 173 and/or the oil centrifuge 186 can be configured to continuously or intermittently discharge accumulated solid particles (referred to as “discharges”). As shown in FIG. 1, discharges 187 from oil centrifuge 186 are combined with defatted emulsion 188 in DFE tank 189.

As mentioned above, the present disclosure relates to using one or more stillage compositions for anaerobic digestion.

In some embodiments, a stillage composition can be separated into at least a first fraction and a second fraction before anaerobic digestion. According to the present disclosure, the stillage composition can be separated at least for the purpose of adjusting the suspended solids content of the stream that is fed to a given anaerobic digestion digester. In some embodiments, a stillage composition can also be separated in manner that helps prepare a process stream for subsequent protein removal and/or oil removal. Separating a stillage composition into at least a first fraction and a second fraction can be performed with a separation system that includes one or more solid-liquid separators like solid-liquid separators such as, e.g., decanters 120 discussed above.

Optionally, a bioprocessing facility can include one or more protein separation systems for separating at least a portion of protein from a stillage composition to form at least a protein stream and a protein-reduced stillage composition before anaerobic digestion. In some embodiments, removing protein from a stillage composition prior to anaerobic digestion can recover protein as a valuable co-product while reducing the amount of ammonia that may be generated during “pre-conditioning” and/or methanogenesis during anaerobic digestion. An example of methods and systems for separating protein from a stillage composition is described in U.S. Pub. No. 2020/0140899 (Bootsma), wherein the entirety of said patent publication is incorporated herein by reference.

Optionally, a bioprocessing facility can include one or more evaporator systems for removing moisture from and concentrating one or more stillage compositions and/or one or more anaerobic digestion effluents (e.g., anaerobic digestion liquid effluent and/or ammonia-stripped anaerobic digestion liquid effluent) to form a condensed stream. A non-limiting example of evaporation system includes an evaporator train as discussed above with respect to FIG. 1.

Optionally, a bioprocessing facility can include one or more oil-removal systems for removing oil from one or more stillage compositions prior to anaerobic digestion. A non-limiting example of an oil-removal system is discussed above with respect to “skim” centrifuge 173 and oil centrifuge 186 in FIG. 1 to form corn oil product 192. In some embodiments, removing oil from a stillage composition prior to anaerobic digestion can recover oil as a valuable co-product while reducing or avoiding any undue impact the oil may have on anerobic digestion.

Optionally, a bioprocessing facility can include one or more pre-conditioning systems to “pre-condition” a stillage composition prior to anaerobic digestion. “Pre-conditioning” involves the breakdown of one or more components (e.g., one or more of carbohydrate, protein, oil and fat, or organic acids, or derivatives of any thereof) in a stillage composition prior to methanogenesis in anerobic digestion. Pre-conditioning can include mechanical, thermal, chemical, and/or biological processes before anaerobic digestion (methanogenesis). For example, pre-conditioning can include one or more of fermentation, hydrolysis, acidogenesis, or acetogenesis. Hydrolysis involves breaking complex organic materials (e.g., carbohydrates, proteins, fats) into simpler soluble compounds (e.g., sugars, amino acids, fatty acids). In some embodiments, hydrolytic bacteria, such as species from genera like, e.g., Clostridium and Bacteroides, can be used for hydrolysis. During acidogenesis, simpler compounds from hydrolysis are converted into volatile fatty acids (e.g., acetic acid, propionic acid, butyric acid), alcohols, hydrogen (H₂), and/or carbon dioxide (CO₂). In some embodiments, acidogenic (fermentative) bacteria, such as species from genera like, e.g., Lactobacillus and Escherichia, can be used for acidogenesis. During acetogenesis, volatile fatty acids (e.g., propionic acid, butyric acid) and alcohols can be converted into acetic acid, hydrogen, and/or carbon dioxide. In some embodiments, acetogenic bacteria, such as, e.g., Syntrophobacter and Acetobacterium, can be used for acetogenesis. One or more pre-conditioning agents can be added to a stillage composition to facilitate pre-conditioning. Non-limiting examples of preconditioning agents include one or more acids, one or more enzymes, one or more consolidated bioprocessing (CBP) yeast, one or more bacteria, and combinations thereof. The primary purpose of pre-conditioning is to prepare feedstock for efficient anaerobic digestion, not to form methane. The environment during pre-conditioning tends not to favor methanogenesis by methanogenic organisms and can be referred to as “non-methanogenic conditions.” That said, some methane may be produced during pre-conditioning. While not being bound by theory, it is believed in some embodiments some microorganisms added to pre-conditioning may produce a relatively small amount of methane. For example, one percent or less by total volume of methane may be produced during pre-conditioning. In some embodiments, no detectable methane is produced during pre-conditioning. An example of preconditioning prior to methanogenesis is described in U.S. Pat. No. 10,590,439 (Ross et al.), wherein the entirety of said patent is incorporated herein by reference.

In some embodiments, one or more non-stillage composition, biomass substrates can be combined with one or more stillage compositions for co-digestion. Adding such biomass substrates can facilitate anaerobic digestion as compared to using a stillage composition as the only substrate (mono-digestion). For example, adding biomass substrates can help adjust the carbon-to-nitrogen (C/N) ratio for improved anaerobic digestion. In some embodiments, adding biomass substrates can produce a C/N ratio of from 20 to 35, or even 20 to 30. Non-limiting examples of biomass substrates include municipal organic waste, lignocellulosic agricultural biomass, industrial waste, and combinations thereof. For example, corn stover can be combined with a stillage composition for co-anaerobic digestion. In some embodiment, the corn stover can be pretreated prior to being combined with the stillage composition. For example, the corn stover can be exposed to a variety of particle-size reduction techniques, thermal treatment, chemical treatment, and the like, prior to being combined with the stillage composition.

Eventually, one or more stillage compositions, or pre-conditioned stillage composition, can be fed to one or more anaerobic digestion digesters. An anaerobic digestion digester is configured to receive at least one feed composition that includes organic matter and expose the feed composition to anaerobic digestion conditions in the presence of one or more methanogenic microorganisms to form an anaerobic digestion digestate composition. An anaerobic digestion digestate composition includes biogas (methane and carbon dioxide) and anaerobic digestion effluent. One or more methanogenic organisms can be added to an anaerobic digestion digester and/or recycled back to the digester after being discharged therefrom.

There are a variety of anaerobic digestion digesters that can be used according to the present disclosure. An anaerobic digestion digester can be classified based on an organic loading that it intended to digest. For example, an anaerobic digestion digester can be classified based on the chemical oxygen demand or “COD” that it can digest or remove.

One class of anaerobic digestion digesters can be referred to as “low organic loading,” which refers to a relatively low rate of COD that is digested. Such “low organic loading” or “low rate” digesters can also be referred to as “high suspended solids” digesters, since a relatively high amount of suspended solids is rate-limiting in terms of how fast the digester can digest the organic loading. In some embodiments, a low organic loading anaerobic digestion digester has an organic loading rate (OLR) of 10 kg COD removal/(m3 reactor volume*day) or less, 5 kg COD removal/(m3 reactor volume*day) or less, 1 kg COD removal/(m3 reactor volume*day) or less, 0.5 kg COD removal/(m3 reactor volume*day) or less, or even 0.1 kg COD removal/(m3 reactor volume*day) or less. Low organic loading digesters can be very large in size. To illustrate this point, to digest 100 kg/day of COD in a stream using a lagoon digester would require a lagoon having a capacity of about 53,000 gallons. In some embodiments, a feed stream to a low organic loading digester has a suspended solids content of 1-20% as-is basis, or even 3-15% as-is basis.

“Low organic loading” digesters can remove at least 50%, from 50-95%, or even from 80-90% of the available BOD in the feed stream in a time period from 7 to 60 days, from 20 to 60 days, or even from 25 to 35 days. Removing available BOD refers to suspended solids being digested to produce biogas by one or more microorganisms.

A non-limiting example of a “low organic loading” digester includes a passive anaerobic digestion digester such as a lagoon (e.g., covered lagoon). “Low organic loading” digesters also include complete mix digesters and plug flow digesters. In “low organic loading” digesters like complete mix digesters and plug flow digesters, the solids retention time (SRT) of the digester, which is the length of time solid particles are held in the digester, equals the hydraulic retention time (HRT), which is the length of time liquid is held in the digester.

A complete mix digester includes a tank that can heat its contents and mix it with an active mass of methane-forming microorganisms. Incoming liquid displaces the digester's present volume, thereby allowing an equal amount of liquid to flow out of the digester.

Methane-forming microorganisms can flow out of the digester along with the displaced liquid. A complete mix digester can be mixed continuously or intermittently mixed.

A plug flow digester is similar to a complete mix digester in that a feed stream flows into the digester and displaces the digester volume, causing an equal amount of material to flow out of the digester. However, the contents of a plug flow digester tends to be thick enough to prevent particles from settling at the bottom. Relatively little mixing occurs in a plug flow digester, thereby causing the contents move through the digester as a plug.

Another class of anaerobic digestion digesters can be referred to as “high organic loading,” which refers to a relatively high rate of COD that is digested. Such “high organic loading” or “high rate” digesters can also be referred to as “low suspended solids” digesters, since a relatively low amount of suspended solids permits such digesters to digest the organic loading relatively faster as compared to a low organic loading digester. In some embodiments, a high organic loading anaerobic digestion digester has an organic loading rate (OLR) of at least 15 kg COD removal/(m3 reactor volume*day), at least 20 kg COD removal/(m3 reactor volume*day), at least 30 kg COD removal/(m3 reactor volume*day), at least 40 kg COD removal/(m3 reactor volume*day), or even at least 50 kg COD removal/(m3 reactor volume*day). High organic loading digesters can be limited by the suspended solids content of the feed stream. In some embodiments, a feed stream to a high organic loading digester has a suspended solids content of 2% or less as-is basis, 1% or less as-is basis, or even 0.5% or less as-is basis.

“High organic loading” digesters can remove at least 50%, from 50-95%, or even from 80-90% of the available BOD in the feed stream in a time period from 1 to 10 days, or even from 1 to 5 days.

Non-limiting examples of “high organic loading” digesters include a hybrid continuous stirred tank reactor (CSTR), anaerobic contact digesters, fixed film digesters, suspended media digesters, sequencing batch reactor digesters, and the like.

Hybrid continuous stirred tank reactors (CSTR) include systems that integrate a CSTR with a membrane bioreactor system such as anaerobic membrane CSTR (ANMBR) and CSTR-AF (anaerobic filter). This combination enables efficient organic matter breakdown in the CSTR followed by membrane filtration in the MBR unit to separate solids and produce high-quality effluent while retaining biological material.

Anaerobic contact digesters are also referred to as contact stabilization digesters and involve recycling solids and returning some of the active methanogenic microorganisms to the anaerobic contact digester to reduce digestion time. It is noted that a plug flow system can achieve a similar advantage by pumping some of the effluent toward the front of the digester, whereas a complete mix digester involves solids settling in an external clarifier before a slurry concentrated in methanogenic microorganism can be recycled back into the complete mix digester.

Fixed film digesters have a column filled with media, such as rings of plastic or wood chips, that methanogenic microorganisms grow on. As a feed stream passes through this media, the media is coated in a growth referred to as biofilm. This type of digester is also referred to as an anaerobic filter or attached growth digester. The retention time for fixed film digesters is relatively short (e.g., less than five days).

Suspended media digesters involve suspending methanogenic microorganisms in a continuous upward flow of liquid. The flow can be adjusted to let smaller particles wash out while keeping larger particles within the digester. The methanogenic microorganisms can create biofilms around the large particles so methanogenic microorganisms remain in the digester. Some suspended media digester designs include a media like sand for microbes to produce biofilm. These digesters are known as fluidized bed digesters. Effluent is sometimes recycled in fluidized bed digesters to help maintain a steady upward flow. Two common types of suspended media digesters are an upflow anaerobic sludge blanket (UASB) digester and an induced blanket reactor (IBR) digester. UASB digesters work well with feed streams having low suspended solids. An expanded granular sludge bed (EGSB) digester is a variant of a UASB digester. Suspended media digesters can digest a similar amount of COD in a relatively much smaller volume as compared to a lagoon. For example, as compared the lagoon example above that required about 53,000 gallons to digest about 100 kg/day of COD in a stream, a UASB digester having a capacity of about 1,500 gallons could digest about 100 kg/day of COD in a stream.

Sequencing batch reactor digesters is an intermittently mixed digester. In an anaerobic sequencing batch reactor (ASBR) digester, methanogenic microorganisms are present in settling solids and decanting liquid. An ASBR digester includes four phases. During the first phase, the ASBR digester is filled. If enough active microbes are present, an ASBR digester may produce biogas with soluble organic liquids. In the second phase, methanogenic microorganisms are mixed together. The third phase follows shortly thereafter and is referred to as a settle stage, which involves the solids being settled. In the fourth phase, effluent is drawn or decanted off. The ASBR digester may repeat the cycle multiple times per day, resulting in almost continuous gas production.

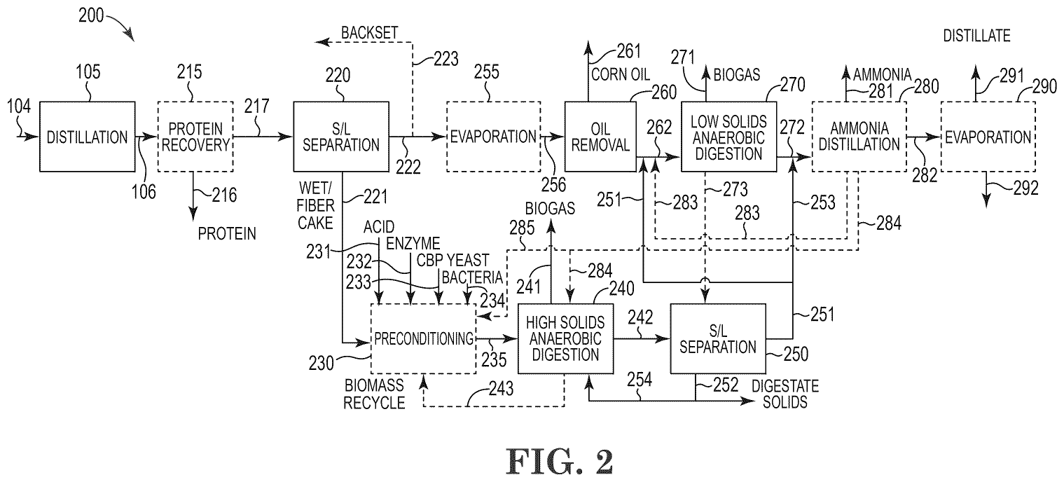

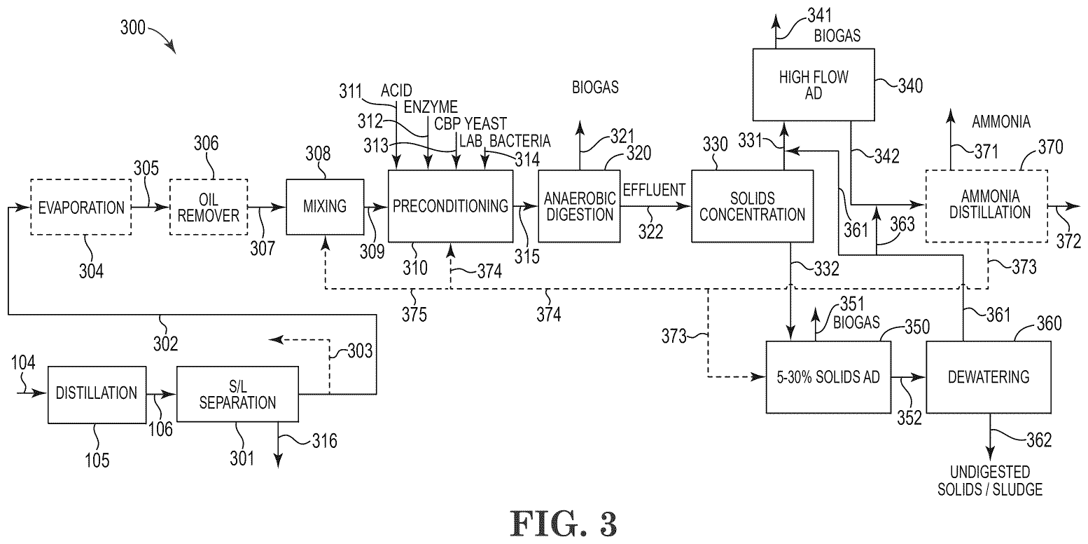

According to the present disclosure, at least one “low organic loading” digester and at least one “high organic loading” digester can be arranged in a variety of series and/or in parallel configurations to achieve one or more advantages as described herein. Non-limiting examples of such configurations are described below with respect to each of FIGS. 2 and 3.

After anaerobic digestion, anaerobic digestion effluent from an anaerobic digestion digester can be separated into at least an anaerobic digestion liquid effluent and an anaerobic digestion solid effluent. A wide variety of separation technologies can be used to separate anaerobic digestion effluent such as membrane separation technologies, density separation technologies, combinations thereof, and the like. The separation technology may be incorporated into an anaerobic digestion digester and/or it may be a subsequent unit operation via a dedicated solid-liquid separator.

In some embodiments, a separation system is in fluid communication with the anaerobic digestion effluent from an anaerobic digestion digester and is configured to receive and separate the anaerobic digestion effluent into at least anaerobic digestion liquid effluent and anaerobic digestion solid effluent. Anaerobic digestion solid effluent has a suspended solids content greater than a suspended solids content of the anaerobic digestion liquid effluent. In some embodiments, a separation system can include one or more solid-liquid separators in series and/or parallel arrangement with respect to each other. Non-limiting examples of solid-liquid separators for separating anaerobic digestion effluent include one or more centrifuges (e.g., two-phase vertical disk-stack centrifuge, three-phase vertical disk-stack centrifuge, filtration centrifuge), one or more decanters (e.g., filtration decanters), one or more filters (e.g., fiber filter, rotary vacuum drum filter, belt filter, filter device having one or more membrane filters), one or more screen devices (e.g., a “DSM” screen (gravity screen); one or more pressure screens; one or more paddle screens; one or more rotary drum screens; one or more centrifugal screeners; one or more linear motion screens; one or more vacu-deck screen; etc.), one or more brush strainers, one or more vibratory separators, one or more hydrocyclones, one or more gravity-settling tanks, one or more presses (one or more screw presses, one or more fan presses), and combinations thereof.

In some embodiments, one or more separation aids may be added to anaerobic digestion effluent to separate the anaerobic digestion effluent into at least anaerobic digestion liquid effluent and anaerobic digestion solid effluent. Non limiting examples of such separation aids include one or more flocculants, one or more coagulants, and combinations thereof.

Optionally, a bioprocessing facility can include one or more ammonia separation systems to separate at least a portion of ammonia from one or more of anaerobic digestion effluent, anaerobic digestion liquid effluent, and anaerobic digestion solid effluent. During anaerobic digestion, organic nitrogen can be converted to ammonia. In some embodiments, it may be desirable to remove ammonia from, e.g., the anaerobic digestion liquid effluent so that an ammonia-stripped anaerobic digestion effluent can be recycled to one or more locations in the bioprocessing facility for use in one or more process streams, thereby helping manage water balance within the bioprocessing facility. Ammonia that is separated can be used as fertilizer, treated via a nitrification-denitrification treatment system, recycled within the bioprocessing facility for pH control of one or more process streams, and the like.

An ammonia separation system can include one or more ammonia separation technologies. One non-limiting example includes an ammonia distillation system, an ammonia crystallization system, an ion selective membrane system, and the like.

In some embodiments, an ammonia distillation system can include a main stripper and a rectifier/side stripper. The three outlet streams from the ammonia distillation system are ammonia-stripped anaerobic digestion effluent (e.g., ammonia-stripped anaerobic digestion liquid effluent) from the bottom of the main stripper, water from the bottom of the rectifier/side stripper, and aqueous ammonia from the top of the rectifier/side stripper.

In some embodiments, the ammonia distillation system may include only two columns (ammonia stripper and ammonia rectifier), however in this configuration ammonia-stripped anaerobic digestion liquid effluent from the bottom of the main stripper would likely be more relatively more dilute, which may require additional evaporation in order to reach the same solid content target.

In some embodiments, the ammonia distillation system may include only one column (ammonia stripper), however in this configuration the ammonia product (either aqueous ammonia or ammonium carbonate and ammonium bicarbonate mixture) would likely be significantly more dilute and require larger systems for storage and/or disposal.

A non-limiting example of a bioprocessing facility according to the present disclosure is described below with respect to FIG. 2. As shown in FIG. 2, bioprocessing facility 200 includes a source of a stillage composition 106 illustrated as whole stillage received from distillation system 105 after distilling a biochemical such as ethanol from beer 104. In some embodiments, whole stillage has a total solids content of 15% or less by weight based on the total weight of the whole stillage on an as-is basis. In some embodiments, whole stillage has a total solids content in a range from 10% to 14%, or even 11% to 13% by weight based on the total weight of the whole stillage on an as-is basis. Total solids is the sum of dissolved solids and suspended solids. Suspended solids refer to material (e.g., corn fiber particles, fat, and the like) that is not dissolved in water that is present in a stillage composition or anaerobic digestion effluent derived from a stillage composition. Non-limiting examples of suspended solids include at least one of plant fiber particles, one or more plant fats (plant oils), one or more proteins, and the like. Protein can include plant protein, cellular protein of one or more microorganisms that are present in a stillage composition (e.g., spent yeast cells), and/or protein produced by one or more microorganisms that are present in a stillage composition. Dissolved solids refer to material that is dissolved in water that is present in a stillage composition. Non-limiting examples of dissolved solids include at least one of one or more of proteins, one or more vitamins, one or more minerals, one or more saccharides, and the like. A non-limiting example of a dissolved protein includes water-soluble corn gluten protein. Percent total solids, percent suspended solids, and percent dissolved solids are reported on an “as-is basis” since moisture is included to describe the degree of concentration of the solids. Components of a stillage composition such as protein can be reported on an as-is or dry-weight basis. Unless noted otherwise, amounts are on a weight basis.

A protein separation system 215 is optionally included to separate protein from stillage composition 106 into protein stream 216, thereby forming a protein-reduced stillage composition 217.

Separation system 220 includes a solid-liquid separator to separate the protein-reduced stillage composition 217 into at least a first fraction 221 (e.g., wet cake) and a second fraction 222 (e.g., thin stillage), where the first fraction 221 has a suspended solids content greater than a suspended solids content of the second fraction 222. Separating the protein-reduced stillage composition 217 in this manner helps concentrate the suspended solids in first fraction 221 and concentrate soluble solids in second fraction 222 so that the first fraction 221 and second fraction can each be directed to an anaerobic digestion digester that anaerobically digests the fractions in a more efficient manner. Optionally, a portion 223 of second fraction 222 can be recycled as backset (e.g., to slurry tank 102) if desired.

In some embodiments, first fraction 221 has a total solids content in a range from greater than 30% to 40% by weight based on the total weight of first fraction 221 on an as-is basis. In some embodiments, the first fraction 221 has a total solids content in a range from 30% to 35% by weight based on the total weight of first fraction 221 on an as-is basis. In some embodiments, the first fraction 221 has a suspended solids content in a range from 15% to 25%, from 15% to 22%, or even from 16% to 18% by weight based on the total weight of first fraction 221 on an as-is basis. In some embodiments, the first fraction 221 has a dissolved solids content in a range from 1% to 5%, from 1% to 3%, or even from 1% to 2% by weight based on the total weight of first fraction 221 on an as-is basis.

In some embodiments, the second fraction 222 has a total solids content in a range from 6% to 12%, from 7% to 11%, or even from 8% to 10% by weight based on the total weight of second fraction 222 on an as-is basis. In some embodiments, the second fraction 222 has a suspended solids content in a range from 4% to 8%, from 5% to 7%, or even from 5% to 6% by weight based on the total weight of second fraction 222 on an as-is basis. In some embodiments, the second fraction 222 has a dissolved solids content in a range from 2% to 4% by weight based on the total weight of second fraction 222 on an as-is basis.

Optionally, as shown in FIG. 2 bioprocessing facility 200 can include a pre-conditioning system 230 that can form pre-conditioned first fraction 235 and enhance the digestibility of pre-conditioned first fraction 235 during subsequent methanogenesis. As shown, one or more pre-conditioning agents can be combined with first fraction 221 to help break down one or more components of the first fraction 221 in the pre-conditioning system 230. Pre-conditioning agents include one or more acids 231, one or more enzymes 232, one or more CBP Yeast, one or more bacteria 234, and combinations thereof. Also, as mentioned above, one or more non-stillage composition, biomass substrates such as corn stover can be added to, e.g., adjust the C/N ration for co-digestion.

Bioprocessing facility 200 includes at least one anaerobic digestion digester 240. If desired, one or more similar anaerobic digestion digesters 240 could be coupled in parallel to accommodate, e.g., relatively large volumetric flows of pre-conditioned first fraction 235. Anaerobic digestion digester 240 is configured to receive and expose the pre-conditioned first fraction 235 to anaerobic digestion conditions to form an anaerobic digestion digestate composition, which includes biogas 241 and anaerobic digestion effluent 242. Because of the relatively high suspended solids in first fraction 221, the anaerobic digestion digester 240 is a “low organic loading,” as described above. In some embodiments, anaerobic digestion would occur in anaerobic digestion digester 240 within 7-60 days, with a conversion of about 50-90% of the BOD.

Optionally, a portion of anaerobic digestion effluent 243 can be recycled to pre-conditioning system 230 to help control the pH of the incoming feed to a pH in a range from 4 to 6.5, which can help promote hydrolytic digestion.

Bioprocessing facility 200 includes a separation system 250. The separation system 250 is configured to receive and separate the anaerobic digestion effluent 242 into anaerobic digestion liquid effluent 251 and anaerobic digestion solid effluent 252. The anaerobic digestion solid effluent 252 includes mostly fibrous solids and has a suspended solids content greater than a suspended solids content of the anaerobic digestion liquid effluent 251. Optionally, a portion 253 of anaerobic digestion liquid effluent 251 can be sent to ammonia distillation system 280, discussed below. Optionally, a portion 254 of anaerobic digestion solid effluent 252 can be recycled to anaerobic digestion digester 240 for further digestion.

Returning to the second fraction 222, after separation system 220, the second fraction 222 goes to an optional evaporation system 255 that removes moisture to form a condensed, second fraction 256. As shown in the FIG. 2, the condensed, second fraction 256 then goes to an optional oil-removal system 260 to separate corn oil 261 and form an oil-reduced second fraction 262.

The oil-reduced second fraction 262 is then sent to anaerobic digestion digester 270, which is a “high organic loading” digester as described above. Also, the anaerobic digestion liquid effluent 251 from anaerobic digestion digester 240 is sent to anaerobic digestion digester 270. The anaerobic digestion liquid effluent 251 is relatively low in suspended solids so it can be exposed to additional digestion by being directly fed to anaerobic digestion digester 270. Optionally, a portion 253 of anaerobic digestion liquid effluent 251 can be sent to ammonia distillation system 280 first to have ammonia removed before sending residual solids in portion 253 through anaerobic digestion digester 270 to help manage ammonia toxicity to methanogenic organisms. Utilizing the “high organic loading” digester for anaerobic digestion digester 270 can be thought of as a secondary “polishing” stage for residual solids in anaerobic digestion liquid effluent 251 that is more efficient and manages water balance better as compared to if the residual solids were simply recycled to anaerobic digestion digester 240, which is a “low organic loading” digester. In some embodiments, “high organic loading” digesters can have 2 to 5 times the organic loading of “low organic loading” digesters. This is because suspended solids take extended time (e.g., 20-60 days) to break down and digest in comparison to the mainly soluble organics in “high organic loading” digesters, where a majority of digestion can take place within 1-10 days. Also, with the use of “high organic loading” digesters there can be a reduction in physical footprint of digesters as “high organic loading” digesters are more efficient on an organic removal rate per gallon of digester volume. Also, concentrating the suspended solids fed to “low organic loading” digesters like anaerobic digestion digester 240 allows for more effective treatment in the pre-conditioning system 230 as the volume of mass flow of material is reduced. This improvement in overall anaerobic digestion can improve digestion efficiency and therefore reduce the retention time needed in the “low organic loading” digesters like anaerobic digestion digester 240.

Anaerobic digestion digester 270 is configured to receive and expose oil-reduced first fraction 262 to anaerobic digestion conditions to form an anaerobic digestion digestate composition that includes biogas 271 and anaerobic digestion effluent 272. In some embodiments, anaerobic digestion would occur in anaerobic digestion digester 270 within 1 to 15 days, with a conversion of about 70-95% of the available BOD. If desired, one or more similar anaerobic digestion digesters 270 could be coupled in parallel to accommodate, e.g., relatively large volumetric flows of oil-reduced first fraction 262, anaerobic digestion liquid effluent 251 from anaerobic digestion digester 240, and/or portion 283 of ammonia-stripped anaerobic digestion effluent 282.

In some embodiments, portion 273 of anaerobic digestion effluent 272 can include excess methanogenic microorganisms (e.g., granulated sludge) can be sent to separation system 250 so that at least a portion of the methanogenic microorganisms can be captured in portion 254 of anaerobic digestion solid effluent 252 that is recycled to anaerobic digestion digester 240 so that the excess methanogenic microorganisms used in anaerobic digestion digester 270 can be utilized to “seed” anaerobic digestion digester 240 with additional methanogenic microorganisms.

As illustrated with FIG. 2, bioprocessing facility 200 includes ammonia separation system 280 that receives anaerobic digestion effluent 272 and portion 253 of anaerobic digestion liquid effluent 251 to separate ammonia 281 and form ammonia-stripped anaerobic digestion effluent 282. Water (not shown) is also separated from anaerobic digestion effluent 272 in ammonia separation system 280. If desired, at least a portion of the water can be recycled to one or more locations in bioprocessing facility 200 as make-up water. For example, at least a portion of the water could be recycled directly back to slurry tank 102 in FIG. 1 as backset.

Optionally, portions 283, 284, and/or 285 of ammonia-stripped anaerobic digestion effluent 282 can be recycled so that the feed streams to anaerobic digestion digester 270, anaerobic digestion digester 240, and pre-conditioning system 230, respectively, e.g., are diluted to control nitrogen/ammonia toxicity therein. Also, a portion (not shown) of ammonia-stripped anaerobic digestion effluent 282 could be recycled directly back to slurry tank 102 in FIG. 1 as make-up water (backset) without any additional processing after ammonia separation system 280.

As illustrated with FIG. 2, bioprocessing facility 200 includes evaporation system 290 that concentrates ammonia-stripped anaerobic digestion effluent 282 by separating water therefrom to form distillate 291 and condensed, ammonia-stripped anaerobic digestion effluent 292. If desired, at least a portion of condensed, ammonia-stripped anaerobic digestion effluent 292 could be reutilized in biorefinery 100 to adjust pH for fermentation.

Another non-limiting embodiment of a bioprocessing facility according to the present disclosure is described below with respect to FIG. 3. As shown in FIG. 3, bioprocessing facility 300 includes a source of a stillage composition 106 illustrated as whole stillage received from distillation system 105 after distilling a biochemical such as ethanol from beer 104.

Separation system 301 includes a solid-liquid separator to separate stillage composition 106 into at least a first fraction 316 (e.g., wet cake) and a second fraction 302 (e.g., thin stillage), where the first fraction has a suspended solids content greater than a suspended solids content of the second fraction 302. Separating the stillage composition 106 in this manner helps concentrate the suspended solids in first fraction and concentrate soluble solids in stillage composition 106. Optionally, a portion 223 of second fraction 222 can be recycled as backset (e.g., to slurry tank 102) if desired.

Optionally, a portion 303 of stillage composition 106 can be recycled as backset.

In some embodiments, stillage composition 106 (shown as whole stillage) has a total solids content of 15% or less by weight based on the total weight of the whole stillage on an as-is basis. In some embodiments, whole stillage has a total solids content in a range from 10% to 14%, or even 11% to 13% by weight based on the total weight of the whole stillage on an as-is basis. Total solids is the sum of dissolved solids and suspended solids. Suspended solids refer to material (e.g., corn fiber particles, fat, and the like) that is not dissolved in water that is present in a stillage composition or anaerobic digestion effluent derived from a stillage composition. Non-limiting examples of suspended solids include at least one of plant fiber particles, one or more plant fats (plant oils), one or more proteins, and the like. Protein can include plant protein, cellular protein of one or more microorganisms that are present in a stillage composition (e.g., spent yeast cells), and/or protein produced by one or more microorganisms that are present in a stillage composition. Dissolved solids refer to material that is dissolved in water that is present in a stillage composition. Non-limiting examples of dissolved solids include at least one of one or more of proteins, one or more vitamins, one or more minerals, one or more saccharides, and the like. A non-limiting example of a dissolved protein includes water-soluble corn gluten protein. Percent total solids, percent suspended solids, and percent dissolved solids are reported on an “as-is basis” since moisture is included to describe the degree of concentration of the solids. Components of a stillage composition such as protein can be reported on an as-is or dry-weight basis. Unless noted otherwise, amounts are on a weight basis.

Returning to the second fraction 302, after separation system 301, the second fraction 302 goes to an optional evaporation system 304 that removes moisture to form a condensed, second fraction 305. As shown in the FIG. 2, the condensed, second fraction 305 then goes to an optional oil-removal system 306 to separate corn oil and form an oil-reduced second fraction 307. The oil-reduced second fraction 307 is then sent to mixing system 308 where oil-reduced second fraction 307 can optionally be combined with 374 of ammonia-stripped anaerobic digestion effluent 375 to adjust the solids (e.g., to about 15-20%) of oil-reduced second fraction 307 and form feed stream 309.

Optionally, as shown in FIG. 3 bioprocessing facility 300 can include a pre-conditioning system 310 that can form pre-conditioned stillage composition 315 and enhance the digestibility of pre-conditioned stillage composition 315 during subsequent methanogenesis. As shown, one or more pre-conditioning agents can be combined with feed stream 309 to help break down one or more components of the feed stream 309 in the pre-conditioning system 310. Pre-conditioning agents include one or more acids 311, one or more enzymes 312, one or more CBP Yeast 313, one or more bacteria 314, and combinations thereof. Also, as mentioned above, one or more non-stillage composition, biomass substrates such as corn stover can be added to, e.g., adjust the C/N ration for co-digestion.

Bioprocessing facility 300 includes at least one anaerobic digestion digester 320. If desired, one or more similar anaerobic digestion digesters 320 could be coupled in parallel to accommodate, e.g., relatively large volumetric flows of pre-conditioned stillage composition 315. Anaerobic digestion digester 320 is configured to receive and expose the pre-conditioned stillage composition 315 to anaerobic digestion conditions to form an anaerobic digestion digestate composition, which includes biogas 321 and anaerobic digestion effluent 322. Because of the relatively high suspended solids in pre-conditioned stillage composition 315, the anaerobic digestion digester 320 is a “low organic loading,” as described above. In some embodiments, anaerobic digestion would occur in anaerobic digestion digester 320 within 10-60 days, with a conversion of about 50-90% of the BOD.

Bioprocessing facility 300 includes a separation system 330. The separation system 330 is configured to receive and separate the anaerobic digestion effluent 322 into anaerobic digestion liquid effluent 331 and anaerobic digestion solid effluent 332. The anaerobic digestion solid effluent 332 includes mostly fibrous solids and has a suspended solids content greater than a suspended solids content of the anaerobic digestion liquid effluent 331.

Anaerobic digestion digester 340 is configured to receive and expose anaerobic digestion liquid effluent 331 to anaerobic digestion conditions to form an anaerobic digestion digestate composition that includes biogas 341 and anaerobic digestion effluent 342. In some embodiments, anaerobic digestion would occur in anaerobic digestion digester 340 within 1 to 15 days, with a conversion of about 70-95% of the available BOD. If desired, one or more similar anaerobic digestion digesters 340 could be coupled in parallel to accommodate, e.g., relatively large volumetric flows of anaerobic digestion liquid effluent 331.

Bioprocessing facility 300 includes at least one anaerobic digestion digester 350. If desired, one or more similar anaerobic digestion digesters 350 could be coupled in parallel to accommodate, e.g., relatively large volumetric flows of anaerobic digestion solid effluent 332. Anaerobic digestion digester 350 is configured to receive and expose the anaerobic digestion solid effluent 332 to anaerobic digestion conditions to form an anaerobic digestion digestate composition, which includes biogas 351 and anaerobic digestion effluent 352. Because of the relatively high suspended solids in anaerobic digestion solid effluent 332, the anaerobic digestion digester 350 is a “low organic loading,” as described above. In some embodiments, anaerobic digestion would occur in anaerobic digestion digester 350 within 7-60 days, with a conversion of about 50-90% of the BOD.

Bioprocessing facility 300 includes a separation system 360. The separation system 360 is configured to receive and separate the anaerobic digestion effluent 252 into anaerobic digestion liquid effluent 361 and anaerobic digestion solid effluent 362. The anaerobic digestion solid effluent 362 (“undigested solids/sludge”) includes mostly fibrous solids and has a suspended solids content greater than a suspended solids content of the anaerobic digestion liquid effluent 361.

The anaerobic digestion liquid effluent 361 from anaerobic digestion digester 350 is sent to anaerobic digestion digester 340. The anaerobic digestion liquid effluent 361 is relatively low in suspended solids so it can be exposed to additional digestion by being directly fed to anaerobic digestion digester 340. Optionally, a portion 363 of anaerobic digestion liquid effluent 361 can be sent to ammonia distillation system 370 first to have ammonia removed before sending residual solids in portion 373 of ammonia-stripped anaerobic digestion effluent 372 to pre-conditioning system 310 to help manage ammonia toxicity. Utilizing the “high organic loading” digester for anaerobic digestion digester 340 can be thought of as a secondary “polishing” stage for residual solids in anaerobic digestion liquid effluent 361 that is more efficient and manages water balance better as compared to if the residual solids were simply recycled to anaerobic digestion digester 350, which is a “low organic loading” digester. In some embodiments, “high organic loading” digesters can have 2 to 5 times the organic loading of “low organic loading” digesters. This is because suspended solids take extended time (e.g., 20-60 days) to break down and digest in comparison to the mainly soluble organics in “high organic loading” digesters, where a majority of digestion can take place within 1-10 days. Also, with the use of “high organic loading” digesters there can be a reduction in physical footprint of digesters as “high organic loading” digesters are more efficient on an organic removal rate per gallon of digester volume. This improvement in overall anaerobic digestion can improve digestion efficiency and therefore reduce the retention time needed in the “low organic loading” digesters like anaerobic digestion digester 350.

As illustrated with FIG. 3, bioprocessing facility 300 includes ammonia separation system 370 (e.g., ammonia distillation) that receives anaerobic digestion effluent 342 and portion 363 of anaerobic digestion liquid effluent 361 to separate ammonia 371 and form ammonia-stripped anaerobic digestion effluent 372. Ammonia 371 is an ammonia-enriched distillate that can be utilized in the bioprocessing facility 300 as, e.g., a micronutrient nitrogen source and/or pH adjustment. Water (not shown) is also separated from anaerobic digestion solid effluent 362 in ammonia separation system 370. If desired, at least a portion of the water can be recycled to one or more locations in bioprocessing facility 300 as make-up water. For example, at least a portion of the water could be recycled directly back to slurry tank 102 in FIG. 1 as backset.

Optionally, portions 373 and 374 of ammonia-stripped anaerobic digestion effluent 372 can be recycled so that the feed streams to anaerobic digestion digester 350 and pre-conditioning system 310, respectively, e.g., are diluted to control nitrogen/ammonia toxicity therein. Also, a portion (not shown) of ammonia-stripped anaerobic digestion effluent 372 could be recycled directly back to slurry tank 102 in FIG. 1 as make-up water (backset) without any additional processing after ammonia separation system 370.

1. A method of producing biogas from one or more stillage streams, wherein the method comprises:
separating a stillage composition into at least a first fraction and a second fraction, wherein the first fraction has a suspended solids content greater than a suspended solids content of the second fraction,
exposing at least a portion of the first fraction to anaerobic digestion conditions in at least one first anaerobic digestion digester to form an anaerobic digestion digestate composition, wherein the anaerobic digestion digestate composition comprises biogas and anaerobic digestion effluent;
separating at least a portion of the anaerobic digestion effluent from the at least one first anaerobic digestion digester into at least anaerobic digestion liquid effluent and anaerobic digestion solid effluent, wherein the anaerobic digestion solid effluent has a suspended solids content greater than a suspended solids content of the anaerobic digestion liquid effluent; exposing at least a portion of the second fraction to anaerobic digestion conditions in at least one second anaerobic digestion digester to form an anaerobic digestion digestate composition, wherein the anaerobic digestion digestate composition comprises biogas and anaerobic digestion effluent, and wherein the at least one second anaerobic digestion digester and the at least one first anaerobic digestion digester are not the same anaerobic digestion digester;
introducing at least a portion of the anaerobic digestion liquid effluent from the at least one first anaerobic digestion digester to the at least one second anaerobic digestion digester; and
exposing the at least a portion of the anaerobic digestion liquid effluent from the at least one first anaerobic digestion digester to anaerobic digestion conditions in the at least one second anaerobic digestion digester to form the anaerobic digestion digestate composition.

2. The method of claim 1, further comprising:
recycling at least a portion of the anaerobic digestion solid effluent from the at least one first anaerobic digestion digester to the at least one second anaerobic digestion digester; and
exposing the at least a portion of the anaerobic digestion solid effluent from the at least one second anaerobic digestion digester to anaerobic digestion conditions in the at least one second anaerobic digestion digester to form the anaerobic digestion digestate composition.

3. The method of claim 1, further comprising separating ammonia (e.g., via distillation) from the anaerobic digestion effluent from the at least one second anaerobic digestion digester to form an ammonia-stripped anaerobic digestion effluent.

4. The method of claim 3, further comprising recycling at least a portion of the ammonia-stripped anaerobic digestion effluent to the at least one first anaerobic digestion digester.

5. The method of claim 3, further comprising recycling at least a portion of the ammonia-stripped anaerobic digestion effluent to the at least one second anaerobic digestion digester.

6. The method of claim 1, wherein separating a stillage composition into at least a first fraction and a second fraction comprises separating the stillage composition via a separation system comprising one or more solid-liquid separators chosen from one or more centrifuges, one or more decanters, one or more filters, one or more screen devices, one or more brush strainers, one or more vibratory separators, one or more hydrocyclones, and combinations thereof.

7. The method of claim 1, wherein the at least one first anaerobic digestion digester comprises one or more anaerobic digestion digesters having an organic loading rate (OLR) of 10 kg COD removal/(m³ reactor volume*day) or less.

8. The method of claim 1, wherein the at least one first anaerobic digestion digester comprises one or more anaerobic digestion digesters chosen from a passive anaerobic digestion digester, a complete mix digester, a plug flow digester, and combinations thereof.

9. The method of claim 1, wherein the at least one second anaerobic digestion digester comprises one or more anaerobic digestion digesters (arranged in series and/or parallel) having an organic loading rate (OLR) of at least 15 kg COD removal/(m³ reactor volume*day).

10. The method of claim 1, wherein the at least one first anaerobic digestion digester comprises one or more anaerobic digestion digesters chosen from a hybrid continuous stirred tank reactor (CSTR) an anaerobic contact digester, a fixed film digester, a suspended media digester, a sequencing batch reactor digester, and combinations thereof.

11. The method of claim 1, wherein separating at least a portion of the anaerobic digestion effluent from the at least one first anaerobic digestion digester into at least anaerobic digestion liquid effluent and anaerobic digestion solid effluent comprises separating the at least a portion of the anaerobic digestion effluent via a separation system comprising one or more one or more solid-liquid separators chosen from one or more centrifuges, one or more decanters, one or more filters, one or more screen devices, one or more brush strainers, one or more vibratory separators, one or more hydrocyclones, and combinations thereof.

12. The method of claim 1, further comprising separating at least a portion of protein from the stillage composition prior to separating the stillage composition into the first fraction and the second fraction.

13. The method of claim 1, further comprising separating at least a portion of oil from the at least a portion of the second fraction prior to exposing the at least a portion of the second fraction to anaerobic digestion conditions in at least one second anaerobic digestion digester.

14. The method of claim 1, further comprising pre-conditioning the at least a portion of the first fraction to at least partially hydrolyze one or more components present in the at least a portion of the first fraction prior to exposing that at least a portion of the first fraction to anaerobic digestion conditions in at least one first anaerobic digestion digester.

15. A bioprocessing facility comprising:
a source of a stillage composition;
a separation system in fluid communication with the source of the stillage composition, wherein the separation system is configured to separate the stillage composition into at least a first fraction and a second fraction, wherein the first fraction has a suspended solids content greater than a suspended solids content of the second fraction;
at least one first anaerobic digestion digester in fluid communication with at least a portion of the first fraction, wherein the at least one first anaerobic digestion digester is configured to receive the at least a portion of the first fraction and expose the at least a portion of the first fraction to anaerobic digestion conditions to form an anaerobic digestion digestate composition, wherein the anaerobic digestion digestate composition comprises biogas and anaerobic digestion effluent;
a separation system in fluid communication with at least a portion of the anaerobic digestion effluent from the at least one first anaerobic digestion digester, wherein the separation system is configured to receive the at least a portion of the anaerobic digestion effluent and separate the at least a portion of the anaerobic digestion effluent from the at least one first anaerobic digestion digester into at least anaerobic digestion liquid effluent and anaerobic digestion solid effluent, wherein the anaerobic digestion solid effluent has a suspended solids content greater than a suspended solids content of the anaerobic digestion liquid effluent; and
at least one second anaerobic digestion digester in fluid communication with at least a portion of the second fraction, wherein the at least one second anaerobic digestion digester is configured to receive the at least a portion of the second fraction and expose the at least a portion of the second fraction to anaerobic digestion conditions to form an anaerobic digestion digestate composition, wherein the anaerobic digestion digestate composition comprises biogas and anaerobic digestion effluent, and wherein the at least one second anaerobic digestion digester and the at least one first anaerobic digestion digester are not the same anaerobic digestion digester, wherein at least a portion of the anaerobic digestion liquid effluent from the at least one first anaerobic digestion digester is in fluid communication with the at least one second anaerobic digestion digester, and wherein the at least one second anaerobic digestion digester is configured to receive and expose the at least a portion of the anaerobic digestion liquid effluent from the at least one first anaerobic digestion digester to anaerobic digestion conditions to form the anaerobic digestion digestate composition.

16. A method of producing biogas from one or more stillage streams, wherein the method comprises:
exposing at least one stillage composition to anaerobic digestion conditions in at least one first anaerobic digestion digester to form an anaerobic digestion digestate composition, wherein the anaerobic digestion digestate composition comprises biogas and anaerobic digestion effluent;
separating at least a portion of the anaerobic digestion effluent from the at least one first anaerobic digestion digester into at least anaerobic digestion liquid effluent and anaerobic digestion solid effluent, wherein the anaerobic digestion solid effluent has a suspended solids content greater than a suspended solids content of the anaerobic digestion liquid effluent;
exposing at least a portion of the anaerobic digestion liquid effluent to anaerobic digestion conditions in at least one second anaerobic digestion digester to form an anaerobic digestion digestate composition, wherein the anaerobic digestion digestate composition comprises biogas and anaerobic digestion effluent;
exposing at least a portion of the anaerobic digestion solid effluent to anaerobic digestion conditions in at least one third anaerobic digestion digester to form an anaerobic digestion digestate composition, wherein the anaerobic digestion digestate composition comprises biogas and anaerobic digestion effluent, wherein the first anaerobic digestion digester, the second anaerobic digestion digester, and the third anaerobic digestion digester are not the same anaerobic digestion digester;
separating at least a portion of the anaerobic digestion effluent from the at least one third anaerobic digestion digester into at least anaerobic digestion liquid effluent and anaerobic digestion solid effluent, wherein the anaerobic digestion solid effluent has a suspended solids content greater than a suspended solids content of the anaerobic digestion liquid effluent;
introducing at least a portion of the anaerobic digestion liquid effluent from the at least one third anaerobic digestion digester to the at least one second anaerobic digestion digester; and
exposing the at least a portion of the anaerobic digestion liquid effluent from the at least one third anaerobic digestion digester to anaerobic digestion conditions in the at least one second anaerobic digestion digester to form the anaerobic digestion digestate composition.

17. The method of claim 16, further comprising:
recycling at least a portion of the anaerobic digestion solid effluent from the at least one third anaerobic digestion digester to the at least one third anaerobic digestion digester; and
exposing the at least a portion of the anaerobic digestion solid effluent from the at least one third anaerobic digestion digester to anaerobic digestion conditions in the at least one third anaerobic digestion digester to form the anaerobic digestion digestate composition.

18. The method of claim 16, further comprising pre-conditioning the at least one stillage composition to at least partially hydrolyze one or more components present in the at least one stillage composition prior to exposing that at least one stillage composition to anaerobic digestion conditions in at least one first anaerobic digestion digester.

19. The method of claim 16, further comprising separating ammonia (e.g., via distillation) from the anaerobic digestion effluent from the at least one second anaerobic digestion digester to form an ammonia-stripped anaerobic digestion effluent.

20. The method of claim 19, further comprising combining at least a portion of the ammonia-stripped anaerobic digestion effluent with the at least one stillage composition prior to and/or while pre-conditioning the at least one stillage composition.

21. The method of claim 19, further comprising combining at least a portion of the ammonia-stripped anaerobic digestion effluent with the at least a portion of the anaerobic digestion solid effluent prior to and/or while exposing the at least a portion of the anaerobic digestion solid effluent to anaerobic digestion conditions in at least one third anaerobic digestion digester.

22. The method of claim 19, wherein separating ammonia from the anaerobic digestion effluent from the at least one second anaerobic digestion digester forms an ammonia-enriched stream.

23. The method of claim 22, wherein the ammonia-enriched stream is used in a bioprocessing facility as a micronutrient-nitrogen source and/or for pH adjustment.

24. A bioprocessing facility comprising:
a source of a stillage composition;
at least one first anaerobic digestion digester in fluid communication with at least a portion of the stillage composition, wherein the at least one first anaerobic digestion digester is configured to receive the at least a portion of the stillage composition and expose the at least a portion of the stillage composition to anaerobic digestion conditions to form an anaerobic digestion digestate composition, wherein the anaerobic digestion digestate composition comprises biogas and anaerobic digestion effluent;
a separation system in fluid communication with at least a portion of the anaerobic digestion effluent from the at least one first anaerobic digestion digester, wherein the separation system is configured to receive the at least a portion of the anaerobic digestion effluent and separate the at least a portion of the anaerobic digestion effluent from the at least one first anaerobic digestion digester into at least anaerobic digestion liquid effluent and anaerobic digestion solid effluent, wherein the anaerobic digestion solid effluent has a suspended solids content greater than a suspended solids content of the anaerobic digestion liquid effluent;
at least one second anaerobic digestion digester in fluid communication with at least a portion of the anaerobic digestion liquid effluent from the separation system, wherein the at least one second anaerobic digestion digester is configured to receive the at least a portion of the anaerobic digestion liquid effluent and expose the at least a portion of the anaerobic digestion liquid effluent to anaerobic digestion conditions to form an anaerobic digestion digestate composition, and wherein the anaerobic digestion digestate composition comprises biogas and anaerobic digestion effluent;
at least one third anaerobic digestion digester in fluid communication with at least a portion of the anaerobic digestion solid effluent from the separation system, wherein the at least one third anaerobic digestion digester is configured to receive the at least a portion of the anaerobic digestion solid effluent and expose the at least a portion of the anaerobic digestion solid effluent to anaerobic digestion conditions to form an anaerobic digestion digestate composition, wherein the anaerobic digestion digestate composition comprises biogas and anaerobic digestion effluent, and wherein the at least one first anaerobic digestion digester, the at least one first anaerobic digestion digester, and the at least one first anaerobic digestion digester are not the same anaerobic digestion digester; and
a separation system in fluid communication with at least a portion of the anaerobic digestion effluent from the at least one third anaerobic digestion digester, wherein the separation system is configured to receive the at least a portion of the anaerobic digestion effluent and separate the at least a portion of the anaerobic digestion effluent from the at least one third anaerobic digestion digester into at least anaerobic digestion liquid effluent and anaerobic digestion solid effluent, wherein the anaerobic digestion solid effluent has a suspended solids content greater than a suspended solids content of the anaerobic digestion liquid effluent, wherein at least a portion of the anaerobic digestion liquid effluent from the at least one third anaerobic digestion digester is in fluid communication with the at least one second anaerobic digestion digester, and wherein the at least one second anaerobic digestion digester is configured to receive and expose the at least a portion of the anaerobic digestion liquid effluent from the at least one third anaerobic digestion digester to anaerobic digestion conditions to form the anaerobic digestion digestate composition.
